(19) United States
(12) Patent Application Publication
(10) Pub. No.: US 2026/0035490 A1
(43) Pub. Date: Feb. 5, 2026

(54) PHOTOPOLYMERIZATION SYNERGIST

(71) Applicant: Piedmont Chemical Industries, LLC, High Point, NC (US)

(72) Inventors: Paul D. Weipert, High Point, NC (US); Bradley A. Hepler, High Point, NC (US); Michael C. Meers, High Point, NC (US); Austin J. Daughtrey, High Point, NC (US)

(21) Appl. No.: 19/053,579

(22) Filed: Feb. 14, 2025

Related U.S. Application Data
(60) Provisional application No. 63/679,256, filed on Aug. 5, 2024.

(51) Int. Cl. C08F 2/50; A61K 8/81; A61Q 3/02; C08F 220/10; C08F 222/10; C09D 133/08

(52) U.S. Cl. CPC C08F 2/50; A61K 8/8152; A61Q 3/02; C08F 220/10; C08F 222/102; C08F 222/103; C09D 133/08; A61K 2800/81

(57) ABSTRACT

A photopolymerization formulation is provided which is particularly suitable for coatings. The formulation comprises a polymerizable ethylenically unsaturated monomer, oligomer or polymer, a photoinitiator a synergist defined by Formula I:
Formula I
wherein R¹, R², R³, R⁴ and X are defined.

### CROSS-REFERENCE TO RELATED APPLICATIONS

This application claims priority to pending U.S. Provisional Patent Appl. No. 63/679,256 filed Aug. 5, 2024 which is incorporated herein by reference.

### FIELD OF THE INVENTION

The present invention is related to improvements in the photopolymerization of monomers and improved photopolymerization formulations. More specifically, the present invention is related to the use of novel synergist which improve the photonic efficiency of a photopolymerization formulation.

### BACKGROUND

Photopolymerization is a well-advanced art which is widely used in the production of materials as diverse as inks, coatings, dental materials, 3-D printing and the like. Though widely practiced, there is an ongoing desire to provide improvements. Most relevant is the desire for improvements in photon efficiency or, simply put, improvements in the number of reacted monomers as a function of the number of photons applied.

The desire for improved photon efficiency is related to manufacturing efficiency the importance of which needs no further explanation. The desire for photon efficiency is also important for increasing the depth of penetration since the photon flux decreases with distance through a photopolymerization formulation. Higher photon efficiency is therefore correlated to the depth of polymerization. Being able to polymerize at deeper depths has significant advantages in 3-D printing applications and allows for the use of thicker coatings when applied to a surface.

In the photopolymerization of unsaturated monomers with UV light, a photoinitiator is used to create a free radical. The free radical produced then begins the polymerization of the unsaturated monomers. In many instances, even after absorption of UV light, the photoinitiator fails to begin the polymerization. In an effort to make the photoinitiators more effective at beginning a polymerization certain compounds have been found to facilitate the formation of a free radical by reacting with the photoinitiator. These compounds are referred to as synergists. Effective synergists allow for the reduction in the amount of the photoinitiator(s) used in the formulation. Commonly used synergists are amines such as N-methyldiethanolamine (MDEA), ethyl-4-dimethylaminobenzoate (EPD) and 2-ethylhexyl-4-dimethylaminobenzoate (EHA). It is thought that these compounds form a free radical on the carbon adjacent to the nitrogen atom.

Though widely used, commonly available synergist still fail to provide adequate photon efficiency. Provided herein are synergist which increase photon efficiency leading to a reduction in the amount of photoinitiator required in a photopolymerization formulation and to increased depth of penetration of polymerization.

### SUMMARY OF THE INVENTION

The present invention is related to improvements in photopolymerization and improved photopolymerization formulations.

More specifically, the present invention is related to improved synergist for photopolymerization.

A particular feature of the invention is the ability to decrease the amount of photoinitiator in a photopolymerization formulation.

Yet another embodiment is provided in a photopolymerization formulation with improved photon efficiency.

These and other embodiments, as will be realized, are provided in a photopolymerization formulation comprising: a polymerizable ethylenically unsaturated monomer, oligomer or polymer; a photoinitiator; and a synergist defined by Formula I:

### Formula I

wherein:
R¹ is a methylene substituted with an aryl, alkene, a cyclic olefin, or a heteroatom containing aromatic ring. R² may be the same as R¹ or branched or straight chain alkyl group of 1 to 12 carbons optionally independently substituted preferably by at least one substituent selected from the group consisting of hydroxyl, amide, carboxyl, ester, trimethoxysilyl and a heteroatom containing aromatic ring. R³ may be the same as R¹ or R² or it may be an oxygen; R⁴ may be the same as R¹ or R²; and X is a counterion preferably selected from the group consisting of halogen; and most preferably chloride; phosphate, sulfate and carbonate.

Yet another embodiment is provided in a method of forming an object comprising:

forming a photopolymerization formulation comprising:
a polymerizable ethylenically unsaturated monomer, oligomer or polymer;
a photoinitiator; and
a synergist defined by Formula I:

### Formula I

wherein:
R¹ is a methylene substituted with an aryl, alkene, a cyclic olefin, or a heteroatom containing aromatic ring. R² may be the same as R¹ or branched or straight chain alkyl group of 1 to 12 carbons optionally independently substituted preferably by at least one of the group consisting of hydroxyl, amide, carboxyl, ester and trimethoxysilyl or a heteroatom containing aromatic ring. R³ may be the same as R¹ or R² or it may be an oxygen; R⁴ may be the same as R¹ or R²; and
X is a counterion preferably selected from the group consisting of halogen; and most preferably chloride; phosphate, sulfate and carbonate; and exposing at least a portion of said photopolymerization formulation to photons.

Yet another embodiment is provided in a road coating formulation comprising: a polymerizable ethylenically unsaturated monomer, oligomer or polymer; a photoinitiator and a synergist defined by Formula I:

### Formula I

wherein: R¹ is a methylene substituted with an aryl, alkene, a cyclic olefin, or a heteroatom containing aromatic ring; R² may be the same as R¹ or branched or straight chain alkyl group of 1 to 12 carbons or a heteroatom containing aromatic ring; R³ may be the same as R¹ or R² or it may be an oxygen; R⁴ may be the same as R¹ or R²; and X is a counterion preferably a halogen; and most preferably chloride; phosphate, sulfate and carbonate.

Yet another embodiment is provided in a method of forming a coating on a road comprising:

forming a photopolymerization formulation comprising:
a polymerizable ethylenically unsaturated monomer, oligomer or polymer;
a photoinitiator; and
a synergist defined by Formula I:

### Formula I

wherein:
R¹ is a methylene substituted with an aryl, alkene, a cyclic olefin, or a heteroatom containing aromatic ring;
R² may be the same as R¹ or branched or straight chain alkyl group of 1 to 12 carbons or a heteroatom containing aromatic ring;
R³ may be the same as R¹ or R² or it may be an oxygen;
R⁴ may be the same as R¹ or R²; and
X is a counterion preferably a halogen; and most preferably chloride; phosphate, sulfate and carbonate; and
exposing at least a portion of the photopolymerization formulation to photons.

Yet another embodiment is provided in a cosmetic nail coating formulation comprising: a polymerizable ethylenically unsaturated monomer, oligomer or polymer; a photoinitiator; and a synergist defined by Formula I:

### Formula I

wherein:
R¹ is a methylene substituted with an aryl, alkene, a cyclic olefin, or a heteroatom containing aromatic ring;
R² may be the same as R¹ or branched or straight chain alkyl group of 1 to 12 carbons or a heteroatom containing aromatic ring;
R³ may be the same as R¹ or R² or it may be an oxygen;
R⁴ may be the same as R¹ or R²; and
X is a counterion preferably a halogen; and most preferably chloride; phosphate, sulfate and carbonate.

Yet another embodiment is provided in a method of forming a cosmetic nail coating comprising:

forming a photopolymerization formulation comprising:
a polymerizable ethylenically unsaturated monomer, oligomer or polymer;
a photoinitiator; and
a synergist defined by Formula I:

### Formula I

wherein:
R¹ is a methylene substituted with an aryl, alkene, a cyclic olefin, or a heteroatom containing aromatic ring;
R² may be the same as R¹ or branched or straight chain alkyl group of 1 to 12 carbons or a heteroatom containing aromatic ring;
R³ may be the same as R¹ or R² or it may be an oxygen;
R⁴ may be the same as R¹ or R²; and
X is a counterion preferably a halogen; and most preferably chloride; phosphate, sulfate and carbonate; and
exposing at least a portion of the photopolymerization formulation to photons.

### DESCRIPTION

The present invention is related to improvements in photopolymerization of monomers. More specifically, the present invention is related to the photopolymerization of ethylenically unsaturated compounds using photoinititators and an inventive synergist.

The photopolymerization process of the instant invention is done in accordance with conventional teachings in the art with the exception of the inclusion of the inventive synergist described herein below. The system employing the photopolymerization is not necessarily limited herein as the inventive synergist can be employed in any system where photopolymerization is commonly used such as, without limit thereto, the curing of coatings, the drying of printing inks, the curing of photo-curable systems such as those used in the formation of printing plates, 3-D printing and various other systems.

The instant invention increases the photoefficiency of the polymerization process, meaning that the number of polymerized monomers per photon of light increases. The instant invention increases the depth of penetration of polymerization thereby allowing for the use of photopolymerization in systems not previously accessible due to the inability of the photons to be of sufficient quantity to polymerize monomers at depth within the photopolymerization formation. The inventive synergist therefore expands the applicability of photopolymerization. In one instance, improved photoefficiency broadens those systems where photopolymerization is applicable. Improving photoefficiency possibly lowers cost which makes previously considered ventures more economically feasible.

Inventive photopolymerization formulations comprise one or more polymerizable ethylenically unsaturated monomers, oligomers or polymers a photoinitiator and an inventive synergist. Other additives may be included as will be further described herein.

Monomers, oligomers or polymers suitable for the polymerization formulation are preferably liquids at ambient temperature, defined herein as 25° C., thereby allowing for the polymerizable formulation to be coated onto or flow over a surface. Alternatively, the monomers, oligomers or polymers may be dissolved in any solvent typically used for the formation of a coating on a substrate or surface.

Specifically suitable for demonstration of the invention are polymerizable ethylenically unsaturated monomers, oligomers or polymers selected from the group consisting of acrylates and methacrylates such as methyl acrylate, ethyl acrylate, propyl acrylate, hexyl acrylate, isooctyl acrylate, methyl methacrylate, ethyl methacrylate, propyl methacrylate, hexyl methacrylate, isooctyl methacrylate, neopentyl diacrylate, trimethylolpropane triacrylate, glycerol triacrylate, tripropylene glycol diacrylate, isobornyl acrylate and the like; acrylonitrile; methacrylonitrile; vinyl esters such as vinyl acetate, vinyl propionate, vinyl acrylate and the like; styrene; divinylbenzene; vinyl chloride; N-vinylpyrrolidone; dialkyl phthalate; dialkyl maleate; ethylene glycol dialkyl ether; thermoplastic resins containing ethylenically unsaturated groups such as unsaturated polyesters derived from fumaric acid, maleic acid and citraconic acid and the like; ethylenically unsaturated dicarboxylic acids; acrylic resins; isocyanate-modified or epoxy-modified resins and the like.

In the formation of a polymerized product or coating the photopolymerization formulation is applied to a substrate or surface followed by exposure to light either simultaneously over the entire sample or specifically over a select portion of the sample. Those portions of monomer, oligomer or polymer exposed to the light polymerize whereas unexposed monomer, oligomer or polymer remain unchanged.

Light sources suitable for demonstration of the invention are not limited but conventional light sources include mercury vapor lamps, fluorescent light sources and laser light sources able to provide wavelengths in the 10-780 nm range. A light source with a significant emission with wavelengths in the ultraviolet to visible portion of the electromagnetic spectrum is preferred with a preference to shorter wavelengths such as 10 to 200 nm.

The term “photoinitiator” is used herein in accordance with the conventionally accepted meaning in the art to refer to an agent which renders a molecule, preferably a polymerizable ethylenically unsaturated monomer, sensitive to the action of light to initiate polymerization wherein the molecule is not otherwise sensitive to light.

Photoinitiators are not particularly limited herein. A photoinitiator initiates polymerization of monomers in response to visible, UV, and/or far-UV wavelengths of light. Examplary photoiniators include oxime-based compounds, triazine-based compounds, benzoin-based compound, acetophenone-based compounds, xanthone-based compounds, and imidazole-based compounds without limit thereto. Photoinitiators particularly suitable for demonstration of the invention include oxime-based compounds such as 1-[4-(Phenylthio)phenyl]-1,2-octanedione 2-(O-benzoyloxime) (OXE-01) or 1-[9-Ethyl-6-(2-methylbenzoyl)-9H-carbazol-3-yl]-ethanone-1-(O-acetyloxime) (OXE-02) available from BASF SE; triazine-based compounds such as 2,4-bistrichloromethyl-6-p-methoxystyryl-s-triazine, 2-p-methoxystyryl-4,6-bistrichloromethyl-s-triazine, 2,4-trichloromethyl-6-triazine, or 2,4-trichloromethyl-4-methylnaphthyl-6-triazine; benzoin-based compounds such as benzophenone, 4-phenylbenzophenone, or p-(diethylamino) benzophenone; acetophenone-based compounds such as 2,2-dichloro-4-phenoxyacetophenone, 2,2-diethoxyacetophenone, 2,2-dibutoxyacetophenone, 2-hydroxy-2-methylpropiophenone, or p-t-butyltrichloroacetophenone; xanthone-based compounds such as xanthone, thioxanthone, 2-methylthio xanthone, 2-isobutylthioxanthone, 2-dodecylthioxanthone, 2,4-dimethylthioxanthone, or 2,4-diethylthioxanthone; and imidazole-based compounds such as 2,2-bis-2-chlorophenyl-4,5,4,5-tetraphenyl-2-1,2-bisimidazole or 2,2-bis (2,4,6- tricyanophenyl)-4,4,5,5-tetraphenyl-1,2-bisimidazole; ketone types such as 2,2-Dimethoxy-2-phenylacetophenone, 1-hydroxycyclohexylphenylketone, or methyl-o-benzoyl-benzoate; phosphorous based compounds such as 2,4,6-trimethylbenzoyldiphenylphosphine oxide, or ethyl (2,4,6-trimethylbenzoyl) phenylphosphinate.

The photopolymerization formulation comprises 1-10 wt % photoinitiator, 85-98.8 wt % polymerizable ethylenically unsaturated monomer(s), oligomer or polymer and 0.2-5 wt % synergist and preferably 0.5 to 5 wt % synergist, optionally in a solvent further optionally comprising at least one additive.

The photopolymerization formulation may further contain up to 13 wt % additives as known in the art, not otherwise altered herein, such as inhibitors to prevent premature polymerization, antioxidants, fillers, pigments or other colorants, thickeners, rheology modifiers and the like without limit thereto. The present invention is particularly suitable when used with fillers and pigments which can absorb or scatter light which decreases the effectiveness of the photoinitiator. By enhancing the photopolymerization the negative impact of fillers and pigments or colorants can be mitigated.

Applications utilizing brightly colored or reflective pigments and other colorants are particularly improved by the present invention. Applications where the instant invention is of particular importance include surface coating applications such as road marking applications where reflective coating, and brightly colored pigments, or other colorants, are of particular interest. Another application is for decorative coatings on fingernails and toenails such as those products referred to as nail polish or gel tip.

A particular advantage of the inventive photopolymerization formulation is that the amount of photoinitiator can be reduced relative to conventional formulations. Another advantage is that the inventive synergists are effective in water dispersions of acrylate resins.

The inventive synergists is effective with hydrogen abstraction types of photoinitiator, such as benzophenone and when used with photoinitiators that function by homolytic fragmentation. The inventive synergist works well when used in conjunction with the typical prior art amine synergists. Inventive synergist are represented by:

### Formula I

wherein:
R¹ is selected from a methylene substituted with an aryl, alkene, a cyclic olefin, or a heteroatom containing aromatic ring. R² may be the same as R¹ or branched or straight chain alkyl group of 1 to 12 carbons optionally independently substituted preferably by at least one of the group consisting of hydroxyl, amide, carboxyl, ester and
trimethoxysilyl or a heteroatom containing aromatic ring. R³ may be the same as R¹ or R² or it may be an oxygen; R⁴ may be the same as R¹ or R²; and
X is a counterion preferably selected from the group consisting of halogen; and most preferably chloride; phosphate, sulfate and carbonate.

For the purpose of the present disclosure a quaternary ammonium synergist has four carbon bonds to the nitrogen wherein at least one carbon-nitrogen bond can be a double bond.

Particularly preferred synergist are represented by compounds below wherein compounds 1, 2 and 17 are controls and the reference numbers are referred to in the examples. The concentration of the compounds is given with the structure and the concentrations used in the following results are adjusted to 100% activity.

### General Preparation of N-Benzyl Quaternary Ammonium Chloride Compounds

The tertiary amine compound was dispersed in water with agitation and heated to 80° C. Room temperature benzyl chloride at 0.97 equivalents was added slowly while maintaining the reaction temperature at 80-85° C. The reaction mixture was held until the amine was converted to the quaternary ammonium compound. They were be tested without further purification. This procedure was used for compounds (3), (7), (8), (9), (10), (11), and (12).

Compound (6) was prepared by reacting benzyldimethylamine in methanol with chloropropyltrimethoxysilane at 105-110° C. for 12 hrs. in a pressure capable autoclave.

Compound (14) was prepared by reacting benzyldimethylamine with an aqueous solution of sodium monochloroacetate at 90° C. for 4 hrs.

Compound (16) was prepared by reacting benzyldimethylamine in water with hydrogen peroxide 33% at 65-75° C. for 5 hrs.

Compound (18) was prepared by reacting benzyldimethylamine in water with dimethylsulfate at 80-85° C. for 3 hrs.

All other chemicals were used as received from commercial sources.

Multiple experiments were conducted to demonstrate the synergistic effect and to demonstrate a reduction in the amount of photoinitiator 1-hydroxycyclohexylphenylketone (CPK) necessary. For the following experiments all samples were exposed by transiting through a Fusion UV Systems F300/F305 at a speed of 0.4 m/min with a coating thickness of 10 mil. The light source was an LED at 385nm. Film cure was judged by measuring pencil hardness and mandrel bend.

Table 1 shows the photopolymerization formulation that was used to determine the synergists' capability.

TABLE 1
100% Solids Acrylate

| | | | Amount |
|---|---|---|---|
| Name | Description | Function | (% wt.) |
| CN 549 | Low viscosity Acrylic | Reactive diluent | 40 |
| | Oligomer/Acrylic Ester | | |
| | blend (Sartomer) | | |
| Ebecryl | Diacrylate ester of Biphenol | Diacrylate Ester of | 40 |
| 600 | A Epoxy Resin (Allnex) | Bisphenol A | |
| SR-351 | TMPTA (Sartomer) | TMPTA | 20 |

Table 2 shows the results of photopolymerization using the inventive compounds.

TABLE 2

| | CPK | | | | Elongation | Pencil | Cure |
|---|---|---|---|---|---|---|---|
| Sample | Conc. | Compound | Conc. | Mandrel | % | Hardness | (P/F) |
| 1 | 2.0% | | | ND | ND | <6B | Fail |
| 2 | 4.0% | | | ¾″ | 7 | H | Pass |
| 3 | 2.0% | (1) Control | 2.0% | ⅝″ | 8.8 | HB | Pass |
| 4 | 2.0% | (1) Control | 1.5% | ¾″ | 7 | 6H | Pass |
| 5 | 2.0% | (1) Control | 1.0% | ¾″ | 7 | F | Pass |
| 6 | 1.0% | (1) Control | 1.0% | ¾″ | 7 | 7H | Pass |
| 7 | 1.0% | (2) Control | 1.0% | 1″ | 5.4 | 5H | Pass |
| 8 | 1.0% | (3) | 1.0% | 1″ | 5.4 | 6H | Pass |
| 9 | 1.0% | (4) | 0.5% | ½″ | 10.6 | 6H | Pass |
| 10 | 1.0% | (5) | 1.0% | 1″ | 5.4 | 6H | Pass |
| 11 | 1.0% | (5) | 0.5% | 1″ | 5.4 | 5H | Pass |
| 12 | 1.0% | (6) | 1.0% | ¾″ | 7 | 5H | Pass |
| 13 | 1.0% | (7) | 1.0% | 1″ | 5.4 | 4H | Pass |
| 14 | 1.0% | (7) | 2.0% | 1″ | 5.4 | 4H | Pass |
| 15 | 1.0% | (8) | 1.0% | ND | ND | <6B | Fail |
| 16 | 1.0% | (9) | 1.0% | ¾″ | 7 | 6H | Pass |
| 17 | 1.0% | (10) | 1.0% | 1″ | 5.4 | 2H | Pass |
| 18 | 1.0% | (11) | 1.0% | ½″ | 10.6 | 6H | Pass |
| 19 | 1.0% | (12) | 1.0% | 1″ | 5.4 | 6H | Pass |
| 20 | 1.0% | (13) | 1.0% | ¾″ | 7 | 5H | Pass |
| 21 | 1.0% | (14) | 1.0% | ½″ | 10.6 | 3H | Pass |
| 22 | 1.0% | (16) | 1.0% | ⅝″ | 8.8 | 8H | Pass |
| 23 | 1.0% | (18) | 0.5% | ⅝″ | 8.8 | 6H | Pass |

As can be seen in samples 1 and 2 in Table 2, the test formulation in Table 1 requires 4% of the photoinitiator CPK to create a film of only moderate pencil hardness. Inventive compounds in samples 8-14 and 16-23 are able to create films with superior properties using only 1% of CPK and 0.5-2.0% of the inventive compounds. This is a 75% reduction in the photoinitiator concentration. These inventive compounds show a parity with compounds previously reported in U.S. Pat. No. 11,421,049 when compared to samples 3-7 which are presented as control samples. The comparable results from samples 9 and 23 demonstrate that the counter ion to the quaternary ammonium compound does not have a large effect on the results.

Table 3 shows the photopolymerization formulation that was used to determine the synergists' capability in waterborne acrylate dispersions.

TABLE 3
Waterborne Acrylate

| | | | Amount |
|---|---|---|---|
| Name | Description | Function | (% wt.) |
| LUX 220 | WPU Dispersion (Alberdingk) | WB UV | 79.6 |
| | | Dispersion | |
| BYK-028 | Compound of modified | Defoamer | 0.6 |
| | polysiloxanes, polyether and | | |
| | hydrophobic particles (BYK) | | |
| BYK-346 | polyether-modified | Surfactant | 0.6 |
| | polysiloxane (BYK) | | |
| Dowanol DPM | DPM Glycol Ether (Dow) | Diluent | 2.9 |
| Water (DI) | | Water | 7.8 |
| Rheolate 288 | Polyether polyurethane solution | Rheological | 1 |
| 50% Solution | in water (Elementis) | Additive | |
| Green 7 C | Green 7 Pigment | Pigment | 7.5 |
| Series | (Sun Chemicals) | | |

Multiple experiments were conducted to demonstrate the synergistic effect and to demonstrate a reduction in the amount of photoinitiator phenylbis (2,4,6-trimethylbenzoyl)-phosphine oxide (BAPO) benzophenone (BP) 1.2/1.0 blend necessary. The formulation in Table 3 with the inventive compounds was prepared and a coating of 10 mil. was applied. The panel was dried at 50° C. for 5 minutes. For the following experiments all samples were exposed by transiting through a Fusion UV Systems F300/F305 at a speed of 0.4 m/min. The light source was a mercury/gallium lamp. Film cure was judged by measuring pencil hardness and mandrel bend.

Table 4 shows the results of photopolymerization using the inventive compounds in a pigmented waterborne acrylate dispersion.

TABLE 4

| | BAPO/BP | | | | Elongation | Pencil |
|---|---|---|---|---|---|---|
| Sample | (1.2/1.0) Conc. | Compound | Conc. | Mandrel | % | Hardness |
| 1 | 1.7% | | | ¼″ | 21.1 | 2B |
| 2 | 1.1% | | | ¼″ | 21.1 | 2B |
| 3 | 0.6% | | | ¼″ | 21.1 | 6B |
| 4 | 0.6% | (1) Control | 0.5% | ¼″ | 21.1 | 3B |
| 5 | 0.6% | (1) Control | 1% | ⅜″ | 14 | H |
| 6 | 0.6% | (2) Control | 1% | ½″ | 10.6 | 2H |
| 7 | 0.6% | (3) | 1% | ⅜″ | 14 | 2H |
| 8 | 0.6% | (5) | 1% | ½″ | 10.6 | 4H |
| 9 | 0.6% | (6) | 1% | ½″ | 10.6 | H |
| 10 | 0.6% | (7) | 1% | ½″ | 10.6 | H |
| 11 | 0.6% | (8) | 1% | ½″ | 10.6 | F |
| 12 | 0.6% | (9) | 1% | ½″ | 10.6 | H |
| 13 | 0.6% | (10) | 1% | ½″ | 10.6 | 2H |
| 14 | 0.6% | (11) | 1% | ½″ | 10.6 | 2H |
| 15 | 0.6% | (12) | 1% | ½″ | 10.6 | 5H |
| 16 | 0.6% | (14) | 1% | ¼″ | 21.1 | H |
| 17 | 0.6% | (15) | 1% | ¼″ | 21.1 | 6B |
| 18 | 0.6% | (16) | 1% | ⅜″ | 14 | 2H |
| 19 | 0.6% | (16) | 0.5% | ⅜″ | 14 | 3H |
| 20 | 0.6% | (17) Control | 1% | ½″ | 10.6 | H |

As can be seen in samples 1 and 2 in Table 2, the test formulation in Table 3 requires 1.1-1.7% of the photoinitiator combination to create a film of only moderate pencil hardness. Inventive compounds in samples 7-19 are able to create films with superior properties using only 0.6% of photoinitiator and 0.5-1.0% of the inventive compounds. This is a 65% reduction in the photoinitiator concentration. These inventive compounds show a parity with compounds previously reported in U.S. Pat. No. 11,421,049 when compared to samples 4-6 and 20 which are presented as control examples.

Table 5 shows the photopolymerization formulation that was used to determine the synergists' capability in fingernail polish gel base coat.

TABLE 5
Fingernail Polish

| | | Amount |
|---|---|---|
| Name | Description | (% wt.) |
| CN 1964 | Diurethane | 42 |
| | dimethacrylate (Sartomer) | |
| Ethyl Acetate | Solvent | 15 |
| Ethyl Alcohol | Solvent | 17 |
| Butyl Acetate | Solvent | 5 |
| Heptane | Solvent | 5 |
| 2-hydroxylethyl | Monomer | 5 |
| methacrylate | | |
| 2-hydroxypropyl | Monomer | 5 |
| methacrylate | | |
| Nitrocellulose | Gloss and flow control | 2 |
| Acrylic acid | Monomer | 2 |

Table 6 shows the results of using the inventive compounds in this formulation. This fingernail gel base coat is typically cured using 2,4,6-trimethylbenzoyldiphenyl phosphine oxide (TPO) photoinitiator at 2 wt. %.

For Samples 1-5 of Table 6 the films were prepared by hand mixing powder photoinitiator and synergist into black pigmented UV curable nail polish. Once mixed until powder has thoroughly dissolved, a 10-mil static drawdown bar was used to apply a wet film to a 6″×3″ Q-panel and the panel was place under a Nail Flair UV lamp for gel nails for 90 seconds of exposure. The films were then wiped with acetone and allowed to cure overnight (18 hours) and were then removed from the panel and tested for dry film thickness using a digital caliper micrometer.

TABLE 6

| | | Photo- | | | Cured Film | |
|---|---|---|---|---|---|---|
| | Photo- | initiator | | | Thickness | Cure |
| Sample | initiator | Conc. | Compound | Conc. | (mil) | (P/F) |
| 1 | TPO | 2.0% | | | 6 | Pass |
| 2 | TPO | 1.0% | | | 4.8 | Pass |
| 3 | BDK | 2.0% | | | 3 | Pass |
| 4 | BDK | 1.0% | (16) | 1.0% | 7 | Pass |
| 5 | BDK | 1.0% | (1) Control | 1.0% | 5 | Pass |

As can be seen in samples 4 and 5,the inventive compounds allow for the replacement of TPO photoinitiator with the photoinitiator 2,2-Dimethoxy-2-phenylacetophenone (BDK) at a reduced level.

Table 7 shows the photopolymerization formulation that was used to determine the synergists' capability in a road marking.

TABLE 7
Road Marking

| | | Amount |
|---|---|---|
| Name | Description | (% wt.) |
| Acrysirup | Commercial road marking resin (No | 41.6 |
| XD-3018B | accelerator) (Mitsubishi Chemical) | |
| Bentone 27 | Bentonite Clay (Elementis) | 0.3 |
| RheoBYK 410 | Rheological Modifier (BYK) | 0.3 |
| DisperBYK 163 | Controlled Flocculating | 0.3 |
| | Dispersant (BYK) | |
| Microna 7 | Calcium Carbonate (Microna) | 55.3 |
| Flexiverse Green 7 | Green Pigment (Sun Chemical) | 2 |
| Aerosil 200 | Fumed Silica (Evonik) | 0.2 |

Table 8 shows the results of using the inventive compounds in this formulation. For Samples 1-12 the films were prepared by hand mixing powder (BAPO) photoinitiator and synergist into liquid MMA Paint for 60 seconds until powder has thoroughly dissolved. A 50-mil static drawdown bar was used to apply a wet film to a 6″×3″ Q-panel and the panel was exposed by transiting through a Fusion UV Systems F300/F305 at a speed of 0.4 m/min. The light source was a mercury/gallium lamp. Film cure was rated for hardness by indentation using a fingernail test and through cure by visual inspection.

TABLE 8

| | Photo- | | | Through | | Surface |
|---|---|---|---|---|---|---|
| | Initiator | | | Cure | | Cure |
| Sample | Conc. | Compound | Conc. | Table 9 | Hardness | (P/F) |
| 1 | 10% | | | 1 | 1 | Pass |
| 2 | 6% | (1) Control | 1.0% | 0 | 0 | Fail |
| 3 | 5% | (16) | 0.5% | 2 | 1 | Pass |
| 4 | 6% | (16) | 0.5% | 2 | 1 | Pass |
| 5 | 6% | (16) | 0.75% | 3 | 4 | Pass |
| 6 | 6% | (16) | 1.0% | 4 | 3 | Pass |
| 7 | 6% | (16) | 2.0% | 2 | 2 | Pass |
| 8 | 7% | (16) | 0.5% | 2 | 2 | Pass |
| 9 | 7% | (16) | 0.75% | 5 | 5 | Pass |
| 10 | 7% | (16) | 1% | 5 | 5 | Pass |
| 11 | 7% | (16) | 2% | 2 | 2 | Pass |
| 12 | 8% | (16) | 0.5% | 3 | 3 | Pass |

TABLE 9
Through-cure number scale based on the following:

| | |
|---|---|
| 1 | Surface Cure only |
| 2 | Some residual liquid material remains under film |
| 3 | Through cure with large film defects (wrinkles, blisters, etc) |
| 4 | Through cure with slight film defects (wrinkles, blisters, etc) |
| 5 | Complete cure with no defects |

Fingernail Hardness Scale:

| | |
|---|---|
| 1 | No resistance to deformation |
| 2 | Minimal resistance/easily indented |
| 3 | Slight resistance to deformation |
| 4 | Moderate resistance to deformation/minimal indentation |
| 5 | No deformation/indentation noted |

As can be seen in Table 8, only samples 9 and 10 were able to produce a defect free film at 50-mil. thickness.

The results demonstrate improved photonic efficiency as evidenced by the improved curing depth and the ability to decrease the amount of photoinitiator in the monomer mixture.

Table 10 shows the photopolymerization formulation that was used to determine the synergists' capability in 3D VAT photopolymerization.

TABLE 10
ABS-Like 3D Resin

| | | | Amount |
|---|---|---|---|
| Name | Description | Function | (% wt.) |
| SR 531 | (5-ethyl-1,3-dioxan-5-yl) | Mono-functional | 79.6 |
| | methyl acrylate | monomer | |
| SR 9003B | Propoxylated | Di-functional | 0.6 |
| | neopentylglycol diacrylate | Monomer | |
| CN 9030 | Urethane Acrylate | Oligomer | 0.6 |
| Titanium | White Pigment | Colorant | 7.8 |
| Dioxide | | | |
| Carbon | Black Pigment | Colorant | 1 |
| Black | | | |

Table 11 shows the results of photopolymerization using the inventive compounds in resin 3D VAT photopolymerization. All samples were prepared using an Elegoo Saturn 2 Resin 3D printer. Each sample conformed to the dimensions of ASTM D638 Type IV dog-bones. For each sample 250 g resin was combined with TPO as a photoinitiator and the inventive compound as outlined in Table 11. Layer height for all samples was 0.05 mm with a 0.5 second rest between layers. All samples were rated for cure by physical inspection in accordance with Table 12. For the purpose of this experiment, Sample 13 will serve as a standard formulation.

TABLE 11

| | | Exposure | | | |
|---|---|---|---|---|---|
| | | Time | | | Cure |
| | Photoinitiator | (seconds | | Synergist | Value |
| Sample | Conc. | per layer) | Synergist | Conc. | (Table 12) |
| 1 | 3% | 2.5 | | | 2 |
| 2 | 3% | 1.7 | | | 1 |
| 3 | 3% | 2.5 | 1 | 1% | 3 |
| 4 | 3% | 1.7 | 1 | 1% | 1 |
| 5 | 3% | 2.5 | 16 | 1% | 4 |
| 6 | 3% | 1.7 | 16 | 1% | 1 |
| 7 | 4% | 2.5 | | | 3 |
| 8 | 4% | 1.7 | | | 2 |
| 9 | 4% | 2.5 | 1 | 1% | 4 |
| 10 | 4% | 1.7 | 1 | 1% | 3 |
| 11 | 4% | 2.5 | 16 | 1% | 4 |
| 12 | 4% | 1.7 | 16 | 1% | 3 |
| 13 | 5% | 2.5 | | | 4 |
| 14 | 5% | 1.7 | | | 3 |
| 15 | 5% | 2.5 | 1 | 1% | 5 |
| 16 | 5% | 1.7 | 1 | 1% | 4 |
| 17 | 5% | 2.5 | 16 | 1% | 5 |
| 18 | 5% | 1.7 | 16 | 1% | 4 |

TABLE 12
Through-cure number scale based on the following:

| | |
|---|---|
| 1 | Complete delamination with no integrity |
| 2 | High level of delamination with minimal |
| | integrity |
| 3 | Minimal delamination >75% of piece is in tact |
| 4 | Complete dog-bone with slight defect |
| 5 | Complete dog-bone with no obvious defect |

As can be seen in Table 11, samples 15 and 17 were the only 2 to produce a defect free dog-bone.

The results demonstrate improved photonic efficiency as evidenced by the ability to decrease the amount of photoinitiator in the resin with samples 5, 9, and 11 while having similar performance to the standard formulation.

All percentages related to concentration are wt % unless otherwise stated.

The invention has been described with reference to the preferred embodiments without limit thereto. Additional embodiments and improvements may be realized which are not specifically set forth herein but which are within the scope of the invention as more specifically set forth in the claims appended hereto.

1. A photopolymerization formulation comprising:
a polymerizable ethylenically unsaturated monomer, oligomer or polymer;
a photoinitiator; and
a synergist defined by Formula I:
Formula I
wherein:
R¹ is selected from a methylene substituted with an aryl, alkene, a cyclic olefin, or a heteroatom containing aromatic ring;
R² may be the same as R¹ or branched or straight chain alkyl group of 1 to 12 carbons or a heteroatom containing aromatic ring;
R³ may be the same as R¹ or R² or it may be an oxygen;
R⁴ may be the same as R¹ or R²; and
X is a counterion preferably a halogen; and most preferably chloride; phosphate, sulfate and carbonate.

2. The photopolymerization formulation of claim 1 wherein at least one of R¹, R², R³ and R⁴ independently are substituted.

3. The photopolymerization formulation of claim 1 wherein at least one of R¹, R², R³ and R⁴ independently are by at least one of the group selected from the group consisting of hydroxyl, amide, carboxyl, ester and trimethoxysilyl.

4. The photopolymerization formulation of claim 1 wherein X is selected from the group consisting of a halogen, a phosphate, a sulfate and a carbonate.

5. The photopolymerization formulation of claim 1 wherein said polymerizable ethylenically unsaturated monomer, oligomer or polymer is selected from the group consisting of acrylates and methacrylates; acrylonitrile; methacrylonitrile; vinyl esters; styrene; divinylbenzene; vinyl chloride; N-vinylpyrrolidone; dialkyl phthalate; dialkyl maleate; ethylene glycol dialkyl ether;
thermoplastic resins containing ethylenically unsaturated groups; ethylenically unsaturated dicarboxylic acids; acrylic resins; isocyanate-modified and epoxy-modified resins.

6. The photopolymerization formulation of claim 5 wherein said polymerizable ethylenically unsaturated monomer, oligomer or polymer is selected from the group consisting of methyl acrylate, ethyl acrylate, propyl acrylate, hexyl acrylate, isooctyl acrylate, methyl methacrylate, ethyl methacrylate, propyl methacrylate, hexyl methacrylate, isooctyl methacrylate, neopentyl diacrylate, trimethylolpropane triacrylate, glycerol triacrylate, tripropylene glycol diacrylate, isobornyl acrylate, vinyl acetate, vinyl propionate and vinyl acrylate.

7. The photopolymerization formulation of claim 5 wherein said polymerizable ethylenically unsaturated monomer, oligomer or polymer is selected from the group consisting of unsaturated polyesters derived from fumaric acid, maleic acid and citraconic acid.

8. The photopolymerization formulation of claim 1 comprising 1-10 wt % said photoinitiator; 85-98.8 wt % said polymerizable ethylenically unsaturated monomer, oligomer or polymer and 0.2-5 wt % said synergist.

9. The photopolymerization formulation of claim 1 wherein said said photoinitiator is selected from the group consisting of oxime-based compounds, triazine-based compounds, benzoin-based compound, acetophenone-based compounds, xanthone-based compounds, and imidazole-based compounds.

10. The photopolymerization formulation of claim 9 wherein said photoinitiator is selected from the group consisting of 1-[4-(Phenylthio)phenyl]-1,2-octanedione 2-(O-benzoyloxime), 1-[9-Ethyl-6-(2-methylbenzoyl)-9H-carbazol-3-yl]-ethanone-1-(O-acetyloxime); 2,4-bistrichloromethyl-6-p-methoxystyryl-s-triazine; 2-p-methoxystyryl-4,6-bistrichloromethyl-s-triazine; 2,4-trichloromethyl-6-triazine; 2,4-trichloromethyl-4-methylnaphthyl-6-triazine; benzophenone; 4-phenylbenzophenone; p-(diethylamino) benzophenone; 2,2-dichloro-4-phenoxyacetophenone; 2,2-diethoxyacetophenone; 2,2-dibutoxyacetophenone; 2-hydroxy-2-methylpropiophenone; p-t-butyltrichloroacetophenone; xanthone; thioxanthone; 2-methylthio xanthone; 2-isobutylthioxanthone; 2- dodecylthioxanthone; 2,4-dimethylthioxanthone; 2,4-diethylthioxanthone; 2,2-bis-2-chlorophenyl-4,5,4,5-tetraphenyl-2-1,2-bisimidazole; 2,2-bis (2,4,6-tricyanophenyl)-4,4,5,5-tetraphenyl-1,2-bisimidazole; benzildimethylketal; 1-hydroxycyclohexylphenylketone; methyl-o-benzoyl-benzoate; 2,4,6-trimethylbenzoyldiphenylphosphine oxide; and ethyl (2,4,6-trimethylbenzoyl) phenylphosphinate.

11. The photopolymerization formulation of claim 1 further comprising at least one additive.

12. The photopolymerization formulation of claim 11 wherein said additive is selected from the group consisting of inhibitors, antioxidants, fillers, pigments, thickeners and rheology modifiers.

13. The photopolymerization formulation of claim 1 further comprising at least one compound selected from the group consisting of N-methyldiethanolamine, ethyl-4-dimethylaminobenzoate, and 2-ethylhexyl-4-dimethylaminobenzoate.

14. A method of forming an object comprising:
forming a photopolymerization formulation comprising:
a polymerizable ethylenically unsaturated monomer, oligomer or polymer;
a photoinitiator; and
a synergist defined by Formula I:
Formula I
wherein:
R¹ is selected from a methylene substituted with an aryl, alkene, a cyclic olefin, or a heteroatom containing aromatic ring;
R² may be the same as R¹ or branched or straight chain alkyl group of 1 to 12 carbons or a heteroatom containing aromatic ring;
R³ may be the same as R¹ or R² or it may be an oxygen;
R⁴ may be the same as R¹ or R²; and
X is a counterion preferably a halogen; and most preferably chloride; phosphate, sulfate and carbonate; and
exposing at least a portion of said photopolymerization formulation to photons.

15-33. (canceled)

34. A road coating formulation comprising:
a polymerizable ethylenically unsaturated monomer, oligomer or polymer;
a photoinitiator; and
a synergist defined by Formula I:
Formula I
wherein:
R¹ is selected from a methylene substituted with an aryl, alkene, a cyclic olefin, or a heteroatom containing aromatic ring;
R² may be the same as R¹ or branched or straight chain alkyl group of 1 to 12 carbons or a heteroatom containing aromatic ring;
R³ may be the same as R¹ or R² or it may be an oxygen;
R⁴ may be the same as R¹ or R²; and
X is a counterion preferably a halogen; and most preferably chloride; phosphate, sulfate and carbonate.

35-46. (canceled)

47. A method of forming a coating on a road comprising:
forming a photopolymerization formulation comprising:
a polymerizable ethylenically unsaturated monomer, oligomer or polymer;
a photoinitiator; and
a synergist defined by Formula I:
Formula I
wherein:
R¹ is selected from a methylene substituted with an aryl, alkene, a cyclic olefin, or a heteroatom containing aromatic ring;
R² may be the same as R¹ or branched or straight chain alkyl group of 1 to 12 carbons or a heteroatom containing aromatic ring;
R³ may be the same as R¹ or R² or it may be an oxygen;
R⁴ may be the same as R¹ or R²; and
X is a counterion preferably a halogen; and most preferably chloride; phosphate, sulfate and carbonate;
and
exposing at least a portion of said photopolymerization formulation to photons.

48-66. (canceled)

67. A cosmetic nail coating formulation comprising:
a polymerizable ethylenically unsaturated monomer, oligomer or polymer;
a photoinitiator; and
a synergist defined by Formula I:
Formula I
wherein:
R¹ is selected from a methylene substituted with an aryl, alkene, a cyclic olefin, or a heteroatom containing aromatic ring;
R² may be the same as R¹ or branched or straight chain alkyl group of 1 to 12 carbons or a heteroatom containing aromatic ring;
R³ may be the same as R¹ or R² or it may be an oxygen;
R⁴ may be the same as R¹ or R²; and
X is a counterion preferably a halogen; and most preferably chloride; phosphate, sulfate and carbonate.

68-79. (canceled)

80. A method of forming a cosmetic nail coating comprising:
forming a photopolymerization formulation comprising:
a polymerizable ethylenically unsaturated monomer, oligomer or polymer;
a photoinitiator; and
a synergist defined by Formula I:
Formula I
wherein:
R¹ is selected from a methylene substituted with an aryl, alkene, a cyclic olefin, or a heteroatom containing aromatic ring;
R² may be the same as R¹ or branched or straight chain alkyl group of 1 to 12 carbons or a heteroatom containing aromatic ring;
R³ may be the same as R¹ or R² or it may be an oxygen;
R⁴ may be the same as R¹ or R²; and
X is a counterion preferably a halogen; and most preferably chloride; phosphate, sulfate and carbonate;
and
exposing at least a portion of said photopolymerization formulation to photons.

81-99. (canceled)
